(19) United States
(12) Patent Application Publication
(10) Pub. No.: US 2009/0262888 A1
(43) Pub. Date: Oct. 22, 2009

(54) AIMING METHOD AND DEVICE FOR AN EXTRAORAL RADIOGRAPHIC TECHNIQUE

(75) Inventors: Chia-Hui Chen, Kaohsiung (TW); Yuk-Kwan Chen, Kaohsiung (TW)

Correspondence Address: CONNOLLY BOVE LODGE & HUTZ LLP, 1875 EYE STREET, N.W., SUITE 1100, WASHINGTON, DC, 20006, US

(73) Assignee: KAOHSIUNG MEDICAL UNIVERSITY

(21) Appl. No.: 12/104,877

(22) Filed: Apr. 17, 2008

(51) Int. Cl. A61B 6/14

(52) U.S. Cl. 378/38

(57) ABSTRACT

An aiming method and a device for an extraoral radiographic technique using a pair of length-adjustable indicator rods. Install a circular locator on each to the indicator rod. While keeping at an appropriate angle, hold the two locators closely beside the cheeks of the patient, and then along the hypothetical axis, secure a radioactive-rays emission head onto one of the circular locator. Following this hypothetical axis to the other locator, and secure a film or a sensor. Aim the radioactive-rays emission head along the hypothetical axis while taking the radiographic shots.

### BACKGROUND OF THE INVENTION

(a) Field of the Invention

This invention is related to the techniques and the use of certain equipment for an endodontic diagnosis, in particular, when taking periapical radiograph extraorally for an endodontic diagnosis of a patient.

(b) Description of the Prior Art

It is common practice for a dentist to take periapical radiographs of a patient during an endodontic treatment or to simply gain a better understanding of a patient's situation in general.

However, for some patients with stomatitis or other possible oral illnesses, it is usually difficult for them to endure having films and/or sensors placed intraorally as an enormous pain could be caused, hence, periapical radiographs could not be successfully obtained. Another scenario would be a patient is having muscular dystrophy around the cheek, causing it difficult to place the film or sensor inside the mouth, thus making periapical radiographs impossible to be taken as well.

Dr. Dan Fisher of the Faculty of Dental Medicine at Hebrew University in Jerusalem published an article in 1974 with regard to having a 22-year-old female patient holding the film and placed it against the chin (outside the mouth) while the radiographs were taken. Slightly blurred images of the upper third molar region were obtained through the introduction of such method. Then until 2003, when Dr. Michael E. Newman and Dr. Seymour Friedman also encountered difficulties due to the inabilities of certain patient populations to accept intraoral films and/or sensors, causing problems during endodontic therapies. Therefore, a similar method described in Dr. Fisher's article was considered and adopted. Literally, had the films placed extraorally. As shown in FIG. 1, a picture taken from Attachment 1 which is a copy of the Journal of Endodontics published by The American Association of Endodontists in 2003 (Vol. 29, No. 6 Jun. 2003). In their article, we can see from the photographs taken that the patient actually held the film against the cheek or chin. The operator can only aim the X-ray beam at the film by adjusting the X-ray cone with bare hands and eye-judgment. And note that the film was unable to be held perpendicular to the path of the X-ray beam. As a result, the images taken were usually blurred or simply useless, raising the cost and slowing down the operation.

Apparently, problems described above are being encountered in many places, and no satisfactory method has yet been designed.

### SUMMARY OF THE INVENTION

The primary objective of this invention is to provide an easy-to-use and fast method to take periapical radiographs.

The secondary objective of this invention is to design an easy-to-use instrument needed for accomplishing the primary goal, and also making sure that the images taken will not be blurred.

Another objective of this invention is to provide a method for assembling the instrument for taking periapical radiographs.

This particular project of this invention is aimed at providing a fast and practical solution for problems mentioned above. Also, devise a method and equipment that would enable the X-ray cone, the film and/or the sensor, and the periapical target all on one axis, hence obtaining clear images during an endodontic treatment.

In order to achieve the objectives mentioned above, the periapical radiographic instrument was designed and assembled, and consisting mainly of the following components: a pair of indicator rods, a clip for fixing the radioactive-ray receiving element, a locator for holding the radioactive-ray emission head.

The indicator rod is length-adjustable, and the “first” indicator rod is joined to the “second” indicator rod, so it can slide freely along the “first” indicator rod. Once the desired length is adjusted, the lock is used to fix the length. In other words, the length of the “first” and “second” indicator rods, when joined together, is adjustable, and for best result, the length should be kept within or equal to 12 centimeters.

A clip, as mentioned before, is fixed on the first indicator rod and is used to secure the radioactive-ray receiving element in place such as the radioactive-ray sensing film or a sensor. Furthermore, the sensor can instantly transmit signals generated upon receiving the radioactive-ray to another analyzing processor or computer via a wire or wirelessly. And the clip and the first indicator rod are also kept approximately perpendicular with respect to each other.

While there is the clip and a sensing element placed on the first indicator rod, there is a locator fixed onto the second indicator rod for securing the radioactive-ray emission head and also accurately aiming the rays to the radioactive rays receiving element on the clip. Since the above mentioned combination of the first and second indicator rods can be adjusted between the lengths of 0 to 12 centimeters, thus, the actual distance between the locator on the second indicator rod and the clip on the first can be kept between 8 to 20 centimeters. A better way is to have the locator coupled with a ring-shaped object, so the radioactive-ray emission head can be nicely joined together.

Furthermore, the best relative distance from the center of the locator to the second indicator rod is to be kept between 5 to 12 centimeters. And so in practice, while the instrument is held close to or against the facial or the mandibular regions of a patient, the film or the sensor can be kept at any part of the patient's cheek or chin and obtain one's molar images successfully.

The preferred embodiment, the locator also comprising a pair of alignment unit, this alignment unit preferred has at least one line-of-sight that is paralleled to the radioactive-rays' path from the radioactive-rays emission head when the locator and the radioactive-rays emission head are joined together. If the previously described locator cannot be joined to the radioactive-ray emission head, then it is best to have the rays' path clearly labeled on the outside of the emission head, so the alignment unit of the locator can be used to make sure that it is properly aligned to the line-of-sight.

The following are the steps and methods for assembling the periapical radiographic instrument mentioned in this invention:

First, establish a hypothetical axis, then construct the first indicator rod, also the second indicator rod, which can be slid freely along the first rod once the two are joined together; the indicator rods and the hypothetical axis should be kept paralleled and to a relative distance between 5 to 12 centimeters. Then fix the clip onto the first indicator rod; a radioactive-rays receiving element is to be attached onto the clip while also keeping the radioactive-rays receiving element along the hypothetical axis; Next, construct a locator onto the second indicator rod, while keeping the locator and the clip on the first indicator rod at a distance between 8 to 20 centimeters; have a radioactive-rays emission head kept onto the locator on the second indicator rod, and keep the radioactive-rays emission head on the hypothetical axis mentioned above as well.

In order to increase the accuracy of the radiographs taken, and to make operation of the instrument easier, the above mentioned method preferred also comprising an alignment unit, located on the locator on the second indicator rod. This alignment unit has at least one line-of-sight that is aligned (paralleled) to the hypothetical axis, providing an accurate reference for adjusting the path of the beam from the radioactive-rays emission head. The path of the radioactive rays is also shown on the outside of the radioactive-rays emission head, providing another reference for aligning to the line-of-sight of the alignment unit.

The radioactive-rays receiving element mentioned above is best composed of a radioactive-rays sensitive film or a sensor, or any other effective radiographic image receivers.

Below describes the methods of using the periapical radiographic imaging instrument mentioned in this invention to take periapical radiographs:

First, fix the radioactive-rays sensitive film or a sensor onto the clip; fix the radioactive-rays emission head onto the locator; adjust the angle of the emission head so it is accurately aimed at the film or the sensor on the clip; and adjust the length of the indicator rods so the clip is close to one side of the cheek while keeping the radioactive-rays emission head on the other side of the cheek; then proceed with the taking of radiographs.

The preferred angulation of the X-ray cone and the bite-plane, with reference to a horizontal plane, is between −20° to −30°. For taking upper or even lower images of the teeth, the angulation of the X-ray cone and the bite-plane should be kept between −10° to −15°.

This invention provides a method and the instrument for taking periapical radiographs, enabling such radiography technique to be carried out in an easier and more accurate manner, generating better radiographs of the upper-left and upper-right, or even lower molar regions for better diagnostic references. The instrument of this invention was clinically tested with a series of 12 adult volunteers. And the result of this study as the Attachment 2, had shown that with this specially designed aiming device, more appropriate radiographs were obtained, in a much easier way.

Below is a description of using an actual case, with photographs attached, to further illustrate the aims, the technical aspects, the characteristics, and the effectiveness of the instrument designed in this invention.

### BRIEF DESCRIPTION OF THE DRAWINGS

FIG. 1 is a picture of prior art which taken from Newman and Friedman's article published in 2003, showing how the radiographs were taken by extraoral means.

FIG. 2 is a block diagram describing the preferred innovated embodiment of this invention, and shows the construction steps of the periapical radiographic instrument.

FIG. 3 is a plan view shows the instrument of the preferred embodiment of this invention, and

FIG. 4 is a section view take through the dotted lines 4-4 in FIG. 3.

FIG. 5A is a picture shows how the instrument of this invention is used, taken during an actual clinical session, and FIG. 5B is an X-ray image of the FIG. 5A.

FIG. 6A is similes to FIG. 5A, but shows another angulation of the X-ray shots, and FIG. 6B is an X-ray image of the FIG. 6A.

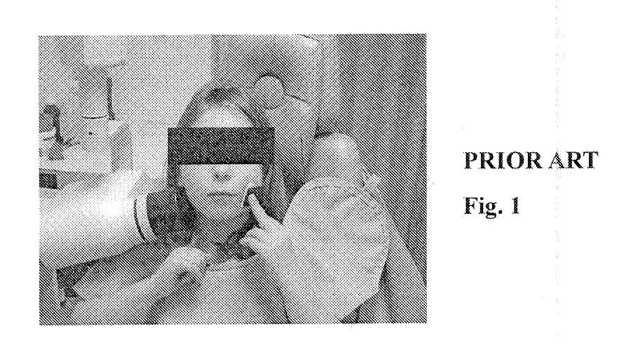

### DETAILED DESCRIPTION OF THE PREFERRED EMBODIMENT

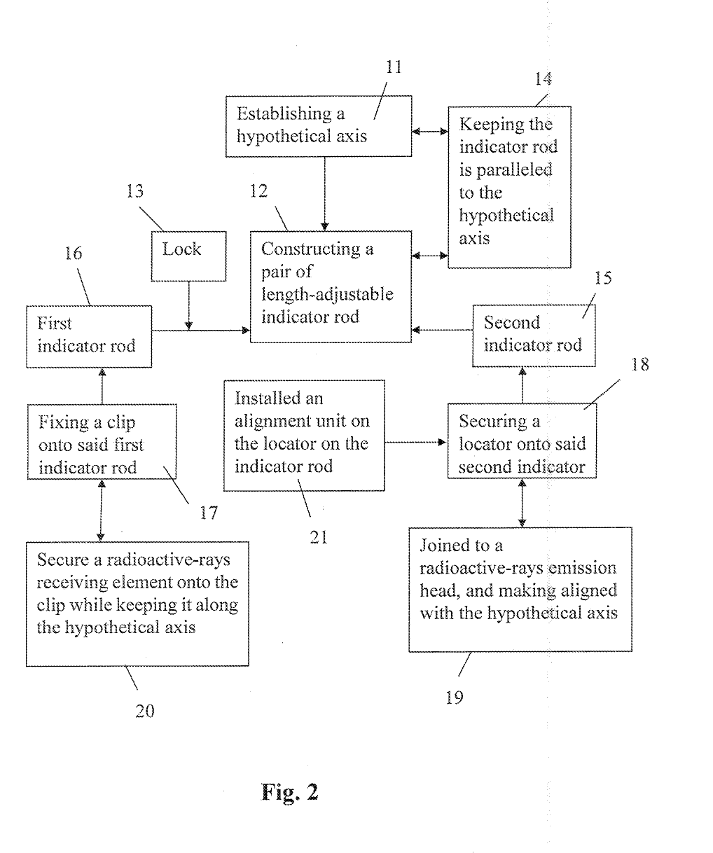

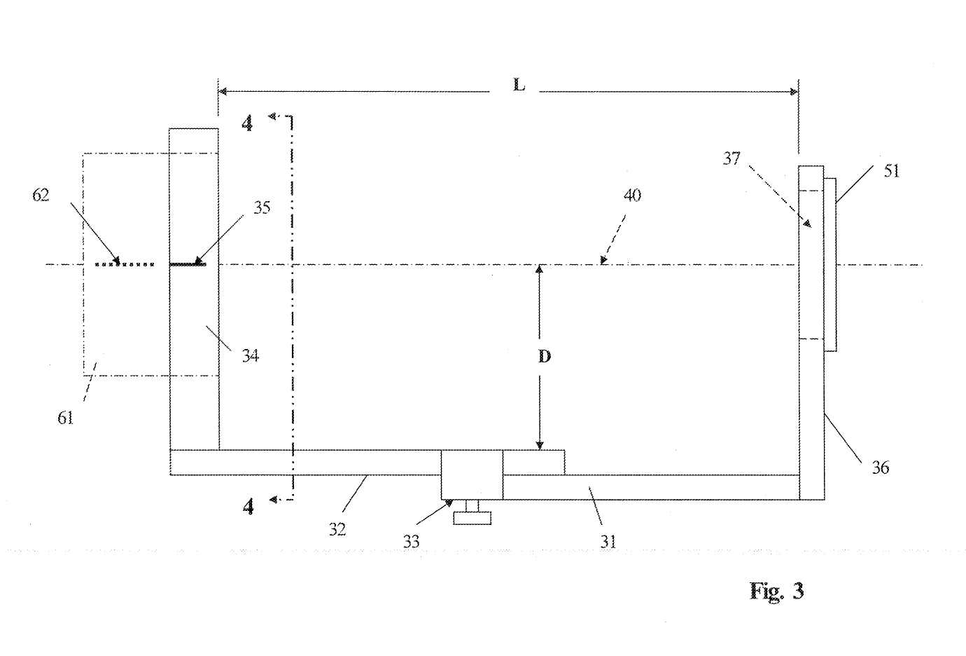

The construction procedures of the periapical radiographic aiming device mentioned in this invention, as shown in FIG. 2, also with reference to FIG. 3, consisting of the following main steps:

First, establish a hypothetical axis (block 11); then construct a pair of length-adjustable indicator rods (block 12); this pair indicator rods is constructed by first, constructing the first indicator rod (block 16) and the second indicator rod (block 15), and then joining the first indicator rod together with the second indicator rod which is able to slide freely along the first indicator rod. The hypothetical axis be aligned with the orientation of indicator rods at all times as could as possible (block 14), and also keeping a distance, D, between the indicator rods and the hypothetical axis, as shown in FIG. 3. This preferred values for distance, D, are between 5 to 12 centimeters so the hypothetical axis can pass through the root areas of the molar teeth of the patient. A lock (block 13) is in place to help secure the indicator rods once the desired length is set.

Fix a clip onto the first indicator rod (block 17); in addition, secure a radioactive-rays receiving element onto this clip while keeping it along the hypothetical axis (block 20). It is better to have a radioactive-ray sensing film or a sensor, or any other radioactive-rays sensitive unit as the radioactive-rays receiving element. Furthermore, construct a locator that is to be fixed onto the second indicator rod (block 18) which is then joined to a radioactive-rays emission head, and making sure that radioactive-rays emission head is also aligned with the hypothetical axis (block 19). The locator on the second indicator rod and the clip on the first indicator rod should be kept at a distance, L. As shown in FIG. 3, the preferred distance, L, should be between 8 to 20 centimeters, hence when the radioactive-rays emission head on the locator is held on one side of the patient's cheek, the radioactive-rays receiving element on the clip can be kept on the opposite cheek.

To further increase the accuracy of the images and also making the operation much easier, an alignment unit is installed on the locator on the second indicator rod (block 21). This alignment unit has at least one line-of-sight that is paralleled with the hypothetical axis, thus enabling easy aiming and adjusting the path of the beams from the radioactive-rays emission head after it is installed. Moreover, the path of the radioactive-rays is shown on the outside of the radioactive-rays emission head, allowing the alignment of the ray emission head and the line-of-sight. Due to the fact that this hypothetical axis is invisible, and the radioactive-rays travel along it reaching to the radioactive-rays receiving element fixed on the other side, therefore this additional line-of-sight labeled on the alignment unit, which is also paralleled to the hypothetical axis, will further enhance the aiming accuracy of the radioactive-rays emission head to the rays receiving element.

The periapical radiographic aiming device described above, as shown in FIGS. 2 and 3, consists of mainly a pair of indicator rods which is constructed using the first indicator rod (31) and the second indicator rod (32), and its length is adjustable. When adjusting the length of the indicator rod, its direction of movement is paralleled to the hypothetical axis (40). The device also consists of the clip (36) used for holding the radioactive-rays receiving element (51), and a locator (34) for holding the radioactive-rays emission head (61) in place.

The indicator rods is preferred further equipped with a lock (33) to secure the indicator rods once the desired length between the first and the second indicator rods is adjusted. In other words, the constructed indicator rod is length-adjustable, and it is best to keep the distance between 0 to 12 centimeters, if so, then the distance, L, of the locator (34) on the second indicator rod (32) and the clip (36) on the first indicator rod (31) can be kept between 8 to 20 centimeters.

The clip (36) is fixed onto the first indicator rod (31), and is for the purpose of securing the radioactive-rays receiving element (51). For example, a radioactive-rays sensing film or a sensor, in addition, a radioactive-rays sensor can instantly transmit signals received to a processor or a computer through a wire or wirelessly. Moreover, the clip (36) and the first indicator rod (31) are kept perpendicular during most of the times, and also has a hole (37) to prevent radioactive signals from weakening before arriving at the radioactive-rays receiving element.

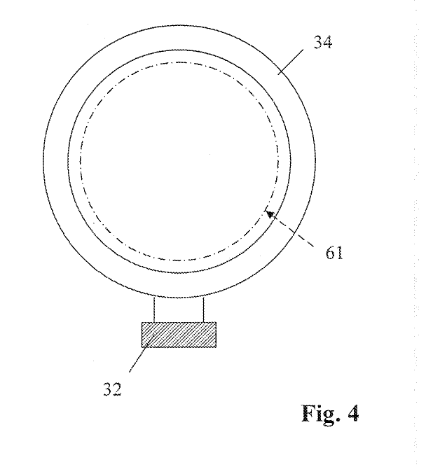

The locator (34) is fixed onto the second indicator rod (32) for the purpose of holding the radioactive-rays emission head (61) and accurately aimed at the radioactive-rays receiving element (51) held by the clip (36). As shown in FIG. 4, the locator (34) preferred is a circular object, thus it can be joined to a radioactive-rays emission head (61).

Also, the distance, D, between the center of the locator (34) and the second indicator rod (32) is preferred kept from 5 to 12 centimeters. This distance, D, in practice will give enough space for keeping the film or the sensor on any part of the cheek when a patient is holding the instrument close to the chin, thus successfully obtain the required molar images.

In addition to the above mentioned instrument, it is preferred also equipped with an alignment unit (35) on the locator (34), such that, from the alignment unit (35), there is at least one line-of-sight, paralleled with the path of the beams from the radioactive-rays emission head (61). If this locator (34) cannot be properly fixed onto the radioactive-rays emission head (61), then it is best to have an indication line (62) shown on the outside of the radioactive-rays emission head (61), so the line-of-sight on the locator (34) can be aimed accurately according to the indication line (62).

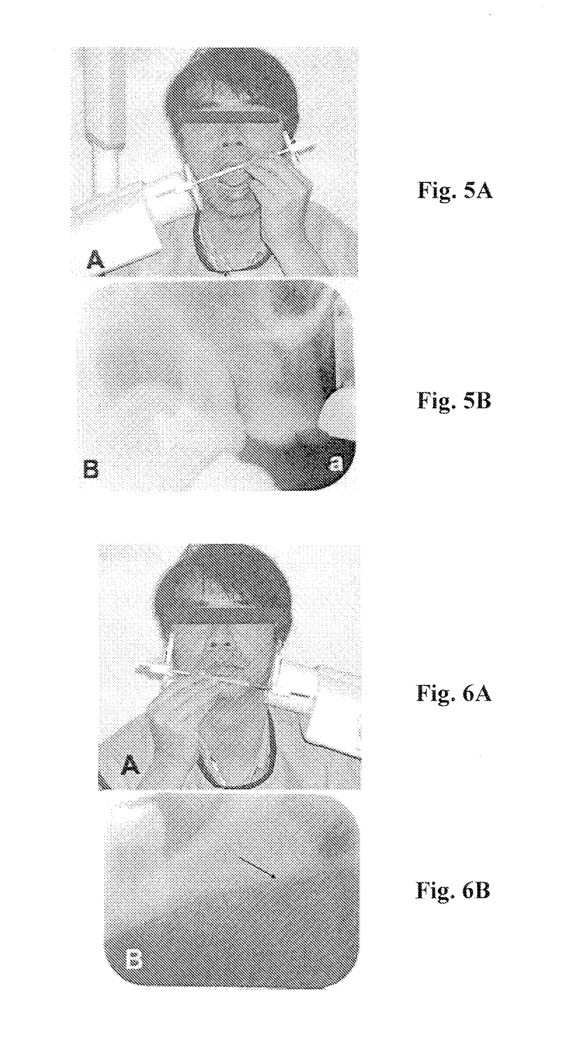

A sample of the mentioned instrument in this invention was constructed. And referring to the photographs as shown in FIG. 5A or FIG. 6A, a periapical radiographic procedure was performed using the aiming device designed by this invention. First, secure a film or a sensor that is sensitive to radioactive-rays onto the clip, and then adjust the length of the indicator rod so the clip is close to the cheek, also keeping the radioactive-rays emission head on the other side. Thus, the radioactive-rays emission head is fixed onto or closely attached to the locator while accurately aimed at the film or the sensor. The X-ray images were taken as shown in FIG. 5B or FIG. 6B, the respective positions when taking the images are shown above the X-ray shots, and the clarity of the X-ray shots are adequate for effective clinical purposes.

According to clinical applications, the angulation between the X-ray cone and the bite-plane, with reference to the horizontal plane, preferred be kept between −20° to −30°. And for taking upper or even lower images of the teeth, the angulation of the X-ray cone and the bite-plane preferred be kept between −10° to −15°.

Although actual and practical experiences from this invention have proven that the design is feasible, but as it is well known to all, there is no restriction on design of this aiming device. Anyone possessing relevant knowledge of such technique is allowed to make slight modification or further improvements, nonetheless, it should not be carried out if the nature of the subject is neglected, and should still be within the scope of the appended claims.

What is claimed:

1. A method for taking periapical radiographic images, comprising the following steps:
establishing a hypothetical axis;
constructing a first indicator rod, and a second indicator rod which can be slide freely along said first indicator rod, together the two said indicator rods will be joined into one length-adjustable indicator rod;
keeping said indicator rod is paralleled to said hypothetical axis, and keeping at a distance from said hypothetical axis of 5 to 12 centimeters;
fixing a clip onto said first indicator rod with a radioactive-ray receiving element attached, and keeping said radioactive-rays receiving element on said hypothetical axis;
constructing and securing a locator onto said second indicator rod, and keeping the distance of said locator on said second indicator rod and said clip on said first indicator rod between 8 to 20 centimeters; and
fixing a radioactive-rays emission head onto said locator that is on said second indicator rod, while also keeping said radioactive-rays emission head aligned with said hypothetical axis.

2. A method according to the claim 1, further comprising the step of constructing an alignment unit on said locator of said second indicator rod, and constructing said alignment unit has at least one line-of-sight that is approximately paralleled to said hypothetical axis, for said radioactive-rays emission head to accurately adjust the path of the beams.

3. A method according to the claim 2, further comprising the step of establishing an indication line shown on the outside of said radioactive-rays emission head, for the purpose of accurately aligning the line-of-sight of said alignment unit.

4. A method according to the claim 1, wherein is using a radioactive rays sensitive film for the radioactive-rays receiving element.

5. A method according to the claim 1, wherein is using a radioactive rays detect sensor for the radioactive-rays receiving element.

6. A periapical radiographic aiming device that comprising:
a pair of length-adjustable indicator rods which consists of a first indicator rod and a second indicator rod that can be freely slid along said first indicator rod;
a clip fixed onto said first indicator rod for holding the radioactive-rays receiving element in place; and
a locator fixed onto said second indicator rod for holding the radioactive-rays emission head in place and also aimed accurately at said radioactive-rays receiving element on said clip; and
wherein the distance between said locator on said second indicator rod and said clip on said first indicator rod be kept at 8 to 20 centimeters.

7. A periapical radiographic aiming device according to the claim 6, further comprising an alignment unit on said locator, and said alignment unit has at least one line-of-sight that is approximately paralleled to the path of the beams that is shooting out from said radioactive-rays emission head.

8. A periapical radiographic aiming device according to the claim 6, further comprising an indication line labeled on the outside of said radioactive-rays emission head, showing the path of the rays.

9. A periapical radiographic aiming device according to the claim 6, wherein said clip and said first indicator rod remain approximately perpendicular with respect to each other.

10. A periapical radiographic aiming device according to the claim 6, wherein the shape of said locator is circular.

11. A periapical radiographic aiming device according to the claim 10, wherein said locator can be fit onto the radioactive-rays emission head.

12. A periapical radiographic aiming device according to the claim 10, wherein the distance between the center of said locator and said second indicator rod be of 5 to 12 centimeters.

13. An application method of using the periapical radiographic aiming device according to the claim 6, comprising the following steps:
fixing a radioactive-rays sensing film or a sensor onto the clip, and fixing the radioactive-rays emission head onto the locator;
aiming said radioactive-rays emission head at said film or said sensor; and
adjusting the length of the indicator rods till having said clip close to one side of the cheek while said radioactive-rays emission head is on the other side, then take the shots.

14. An application method according to the claim 13, wherein keeping the angulations between the X-ray cone and the bite-plane, with respect to a horizontal plane within −20° to −30°.

15. An application method according to the claim 13, wherein keeping the angulations between the X-ray cone and the bite-plane, with respect to a horizontal plane within −10° to −15°.
